(19) United States
(12) Patent Application Publication
(10) Pub. No.: US 2013/0199530 A1
(43) Pub. Date: Aug. 8, 2013

(54) Y CONNECTOR/ADAPTER ALLOWING CONNECTION OF TWO STANDARD OXYGEN SUPPLY TANK INLET TUBES TO FEED TO SINGLE STANDARD SIZE OUTLET TUBE

(71) Applicant: Gina Burger, Edmond, OK (US)

(72) Inventor: Gina Burger, Edmond, OK (US)

(21) Appl. No.: 13/365,196

(22) Filed: Feb. 2, 2012

(51) Int. Cl. A61M 16/00

(52) U.S. Cl. 128/204.18

(57) ABSTRACT

This is directed to systems, processes, machines, and other means that provide increased oxygen flow to medical patients. The invention can combine two oxygen tanks into a specially made adapter that can provide an increased flow of oxygen to medical patients.

### CROSS REFERENCE TO RELATED APPLICATIONS

Not Applicable

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION BY REFERNCE OF MATERIAL SUBMITTED ON A COMPACT DISC

Not Applicable

### FIELD OF THE INVENTION

This device relates to techniques for delivery of high flows of oxygen to a patient in a safe and reliable manner.

### BACKGROUND OF THE INVENTION

Oxygen masks have existed in some rudimentary form since 1847. Traditional oxygen production systems for individuals have used a single mask connected to a single oxygen tank by a single oxygen tube. While this one-size-fits-all approached worked well for many years, new challenges in this field demand a new design, which the present invention discloses. In particular, medically compromised patients require higher oxygen flows as well known science indicates, however, the conventional oxygen supply system provides insufficient flows of oxygen to patients who are trying to move or otherwise perform activities that require a higher flow of oxygen.

Simply retrofitting two plastic tubes with two plastic tubes will not result in the safety and reliability required of medical devices.

### BRIEF SUMMARY OF THE INVENTION

Methods, systems, and other means are provided for providing a greater volume and flow of oxygen for hospital patients with a novel oxygen tank assembly. In accordance with some embodiments an adapter comprising a first end and a second end is disclosed. The first end of the adapter further comprises a first connector and a second connector. The second end further comprises a third connector. The first connector is mechanically coupled to a first oxygen tube by a first linking mechanism. The first oxygen tube is mechanically coupled to a first oxygen bottle. The second connector is mechanically coupled to a second oxygen tube by a second linking mechanism. The second oxygen tube is mechanically coupled to a second oxygen bottle. The third connector is mechanically coupled to an oxygen delivery device (mask or manual resuscitator bag, or similar device) tube by a third linking mechanism. The oxygen delivery device is mechanically coupled to the oxygen tube.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIG. 1: is a perspective view of the invention shown in use.

FIG. 2: is a perspective view of the invention.

FIG. 3: is a side view of the invention.

FIG. 4: is a section view of the invention.

FIG. 5: is a perspective view of a first alternate embodiment of the invention.

FIG. 6: is a perspective detail view of a second alternate embodiment of the invention.

FIG. 7: is a section detail view of the second alternate embodiment of the invention.

### DETAILED DESCRIPTION OF THE INVENTION

Embodiments of the present invention overcome many of the obstacles associated with existing portable oxygen supply systems, and now will be described more fully hereinafter with reference to the accompanying drawings that show some, but not all embodiments of the claimed inventions. Indeed, the invention may be embodied in many different forms and should not be construed as limited to the embodiments set forth herein. Rather, these embodiments are provided so that this disclosure will satisfy applicable legal requirements. Like numbers refer to like elements throughout.

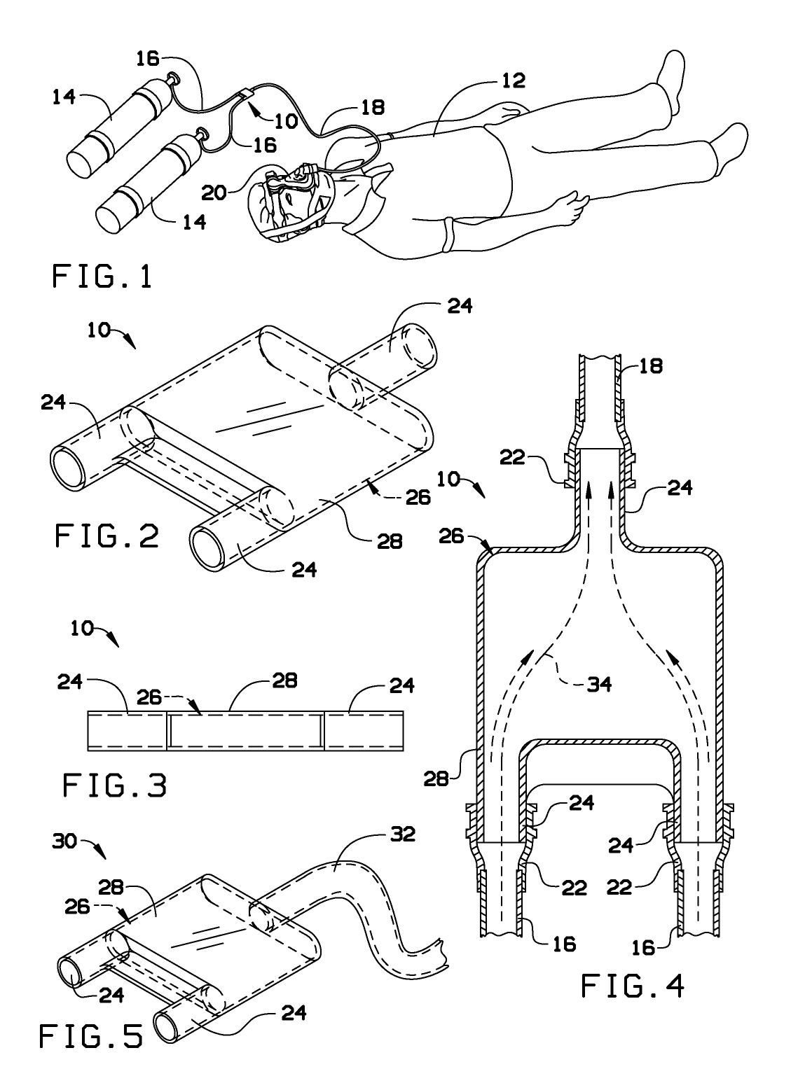

FIG. 1 shows a user with an oxygen tank assembly. User 12 is a medical patient with a need for oxygen, which is stored in a first oxygen tank 14 and a second oxygen tank 14. The first oxygen tank 14 is mechanically coupled to a first oxygen tank tube 16 in a manner well known in this field. Similarly, the second oxygen tank is mechanically coupled to a second oxygen tank tube. Two tank tubes 16 are mechanically coupled to adapter 10 as shown in more detail in FIG. 2 below. Adapter 10 is mechanically coupled to oxygen delivery device tube 18; which is mechanically coupled to oxygen device 20 using well known methods.

FIG. 2 shows a side view of adapter 10. Adapter 10 comprises outer shell 28 and inner shell 26. Outer shell 28 is an outer medical plastic body. Inner shell 26 is an inner medical plastic body. Male adapter 24 is mechanically coupled to shell 28 in three places as shown in FIG. 2. Male adapter 24 is a standard ¼ inch male oxygen adapter, and FIG. 2 shows a first ¼ inch male adapter, a second ¼ inch male adapter and a third ¼ inch male adapter.

FIG. 3 shows a side view of adapter 10. Adapter 10 comprises outer shell 28 and inner shell 26. Outer shell 28 and inner shell 26 are mechanically coupled to a plurality of male adapters 24. For example, this unit can be created by molding hard medical plastic into a single unit.

FIG. 4 shows a section view of adapter 10. As explained above, adapter 10 comprises outer shell 28, inner shell 26 and a plurality of male adapters 24. Male adapter 24 is mechanically coupled to tank tube 16 by female adapter 22 at a first end of adapter 10. Female adapter 22 is the standard ¼″ medical hose tubing with a female adapter which is the first medical hose tubing and the second medical hose tubing. At a second end of adapter 10, is male adapter 24. Male adapter 24 is mechanically coupled to oxygen delivery device tube 18 by female adapter 22 where oxygen delivery device tube can be a third medical hose tubing.

FIG. 5 shows a sectional view of a first alternate embodiment of the invention. Adapter 30 comprises outer shell 28, inner shell 26 and a plurality of male adapters 24. Adapter 30 has two ends with two male adapters 24 at a first end and molded body hose 32 at the opposite end. Here, the body hose is molded to adapter 30 in a single unit. This unit is also used with a pair of oxygen tanks 14 and an oxygen delivery device tube 18 that are arranged in a manner similar to adapter 10 above.

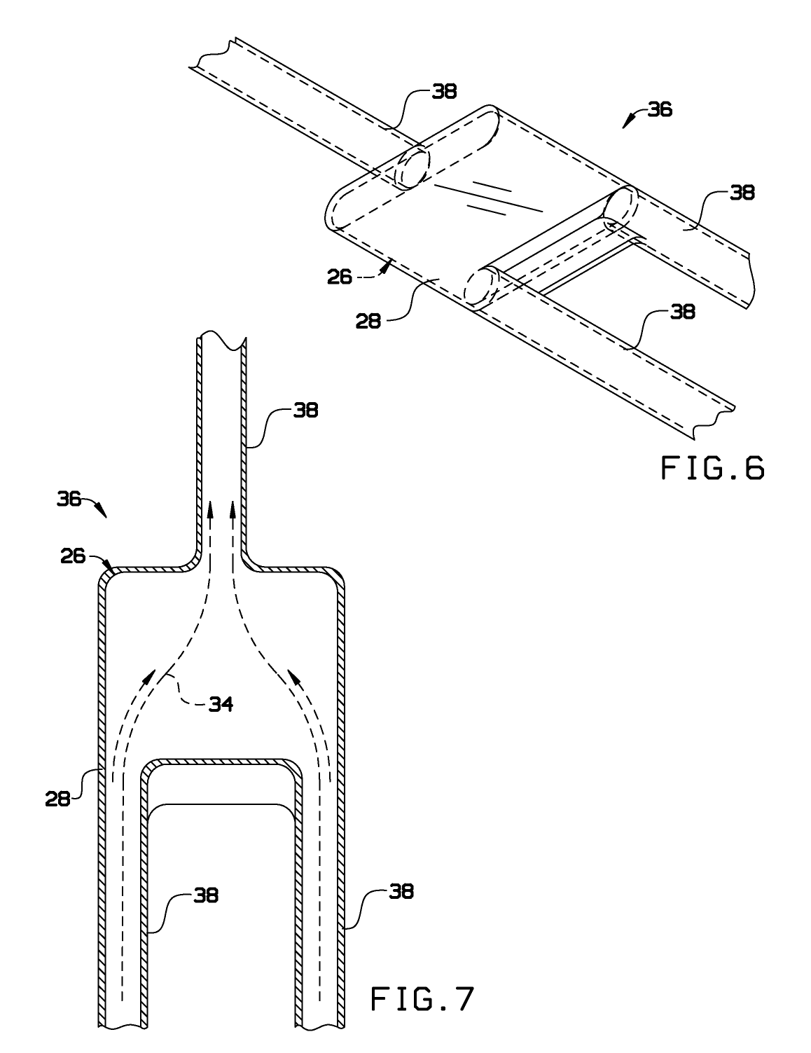

FIG. 6 shows a perspective view of the second alternate embodiment of the invention. Adapter 36 comprises outer shell 28, inner shell 26 and a plurality attached tubing 38. Attached tubing 38 contains a mechanical couple to outer shell 28. The mechanical couple is a press fit seal created by pushing attached tube 38 over outer shell 28 in three places creating a first press fit seal, a second press fit seal and a third press fit seal. This unit is also used with a pair of oxygen tanks 14 and an oxygen delivery device tube 18 that are arranged in a manner similar to adapter 10 above.

FIG. 7 shows a perspective view of the second alternate embodiment of the invention. Adapter 36 comprises outer shell 28, inner shell 26 and a plurality attached tubing 38. Oxygen flow 34 shows the path of oxygen from oxygen tanks 16 (not shown) through adapter 36 to oxygen mask 16 (not shown)

That which is claimed:

1. An oxygen tank assembly comprising
an adapter comprising a first end and a second end
where the first end further comprises a first connector and a second connector; and
where the second end further comprises a third connector
a first oxygen tank mechanically coupled to a first oxygen tank tube;
where the first oxygen tank tube is mechanically coupled to the first connector by a first linking mechanism
a second oxygen tank mechanically coupled to a second oxygen tank tube;
where the second oxygen tank tube is mechanically coupled to the second connector by a second linking mechanism
an oxygen mask mechanically coupled to an oxygen delivery device tube;
where the oxygen delivery device tube is mechanically coupled to the third connector by a third linking mechanism.

2. The adapter of claim 1, further comprising,
the first connector is a first ¼ inch male adapter; the second connector is a second ¼ inch male adapter and the third connector is a third ¼ inch male adapter;
the first linking mechanism is a molded body hose with a first press fit seal; the second linking mechanism is a first medical hose tubing with a second press fit seal and the third linking mechanism is a second medical hose tubing with a third press fit seal.

3. The adapter of claim 1, further comprising,
the first connector is a first ¼ inch male adapter; the second connector is a second ¼ inch male adapter and the third connector is a third ¼ inch male adapter;
the first linking mechanism is first medical hose tubing molded to the first ¼ inch male adapter; the second linking mechanism is a second medical hose tubing molded to the second ¼ inch male adapter and the third linking mechanism is a third medical hose tubing molded to the third ¼ inch male adapter.

4. The adapter of claim 1, further comprising,
the first connector is a first ¼ inch male adapter; the second connector is a second ¼ inch male adapter and the third connector is a third ¼ inch male adapter;
the first linking mechanism is a first medical hose tubing which is press fit onto the first ¼ inch male adapter; the second linking mechanism is a second medical hose tubing which is press fit onto the second ¼ inch male adapter and the third linking mechanism is a third medical hose tubing which is press fit onto the third ¼ inch male adapter.
